(19) United States
(12) Patent Application Publication
(10) Pub. No.: US 2021/0196157 A1
(43) Pub. Date: Jul. 1, 2021

(54) Chemically Fused Membrane for Analyte Sensing

(71) Applicant: Jeff T. Suri, Fallbrook, CA (US)

(72) Inventor: Jeff T. Suri, Fallbrook, CA (US)

(73) Assignee: Glucovation, Inc., Carlsbad, CA (US)

(21) Appl. No.: 17/136,178

(22) Filed: Dec. 29, 2020

Related U.S. Application Data
(60) Provisional application No. 62/954,793, filed on Dec. 30, 2019.

(51) Int. Cl. A61B 5/145; C12Q 1/00; A61B 5/1486

(52) U.S. Cl. CPC A61B 5/14532; C12Q 1/006; C12Q 1/003; C08F 220/18; A61B 2562/0217; A61B 2562/125; A61B 5/14865

(57) ABSTRACT

The invention disclosed herein is a device having an analyte sensor, having a working electrode and a membrane disposed over the electrode and methods of using the device. The multilayered membrane is formed by chemically fusing an inner layer of a polyelectrolyte with an outer layer of an ethylenically unsaturated prepolymer through a chain-growth polymerization reaction.

### CROSS-REFERENCE TO RELATED APPLICATIONS

Not applicable

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATION-BY-REFERENCE OF MATERIAL SUBMITTED ON A COMPACT DISC

Not applicable

### TECHNICAL FIELD

The present invention relates generally to membranes utilized in biological testing and measuring devices. More specifically, the device relates to membranes used with biosensors for the detection and measurement of analytes in biological samples.

### BACKGROUND OF THE INVENTION

Biosensors have become important tools in wearables and self-monitoring devices. Their ability to detect an analyte of interest in vivo and allow for real-time modifications of human behavior and inputs is becoming more important for the development of devices related to personalized medicine and healthcare. Continuous glucose monitoring (CGM) falls into this category. With commercially available products on the market, CGM has proven to be one of the most valuable approaches to managing diabetes. Finger-stick-based glucometers are becoming a tool of the past as CGM products are beginning to take over the market for effective management of diabetes.

Although CGMs have proven to be effective there is still room for technological improvement. Easy-to-use devices still need to be developed that remain accurate over their use-life and that are relatively inexpensive.

One factor that impacts the accuracy and ease of use for CGMs is their stability and requirement for calibration. Calibration is necessary because of either (A) changes that take place in the physiologic environment around the bio sensor or (B) because of changes in the sensor chemistry itself as a function of time or environment. Although dealing with (A) can be challenging and variable, (B) can be addressed via the proper design of the biosensor chemistry.

Electrochemically-based CGMs usually comprise a multilayered membrane with an outer membrane facing the bodily fluid or tissue. While the outer membrane controls the permeation of analyte, the inner membrane, sometimes called the enzyme layer, functions to react with the analyte to produce a product that further reacts with the surface of the electrode. For this type of sensing system, it is critical that the enzyme is properly immobilized within the inner layer. This will ensure stable signal and longevity of the sensor.

There are various approaches to immobilizing the enzyme in the inner layer. For a 1^{st} generation glucose sensor (where there is no mediator) the enzyme layer serves to provide a stable environment for the enzyme to function properly without any inhibition of its active site. It also needs to contain a sufficient quantity of oxygen molecules in order for the sensor output to be proportional to the prevailing glucose concentration in the tissue. All of these requirements are necessary in order avoid signal drift of the device and allow for the accurate measurement of glucose concentrations in vivo without the constant re-calibration of the sensor.

Various groups have addressed these requirements in different ways. U.S. Pat. No. 9,737,250 claims that addition of PVP to the GOX layer improves oxygen permeability and stability of the sensor. It is reported that the addition of one or more hydrophilic polymers in the enzyme layer results in improved sensor performance (i.e., less signal drift) under low oxygen conditions.

U.S. Pat. No. 8,280,474 claims that in order to improve the stability and lifetime of a sensor the Ag/AgCl reference electrode is covered with an impermeable dielectric layer or a permselective coating that decreases the solubility of the AgCl to the surrounding aqueous environment, thereby improving the stability and longevity of the electrochemical sensor.

U.S. Pat. No. 7,090,756 describes the use of trifunctional crosslinkers to help stabilize transition metals for a wired enzyme sensor. Inadequate crosslinking of a redox polymer can result in excessive swelling of the redox polymer and to the leaching of the components. The crosslinkers form a tighter network and inhibit leaching of the metals from the sensor. This provides for a more stable sensor signal.

PCT 2017/189764 describes the use of a glucose oxidase (GOX) bioconjugate that is UV-cured into a polymer matrix to provide more long-term (over 6 days) stability during in vivo sensor operation.

U.S. Pat. No. 866,062 describes the use of multilayered membrane consisting of an electrode layer covered with an analyte sensing layer and an analyte modulating layer that functions in analyte diffusion control. Blends of polyurethane/polyurea and a polymeric acrylate for the analyte sensing layer were found to allow for the ability to eliminate the need for a separate adhesion promoting material disposed between various layers of the sensor (eg one disposed between the analyte sensing layer and the analyte modulating layer). This helped overcome hydration challenges and the sensor's ability to provide accurate signals that correspond to the concentrations of glucose.

U.S. patent application US20140012115A describes the use of adhesion promoting layers in between an analyte sensing layer and an analyte modulating layer in order to address problems associated with sensor layers delaminating and/or degrading over time in a manner that can limit the functional lifetime of the sensor. The AP layers are formed by plasma treatment and hexadimethylsiloxane treatment of the analyte modulating layer.

U.S. Pat. Nos. 6,514,718, 5,773,270, and 4,418,148 describe how the use of a multilayered membrane gives optimal response stability, good mechanical strength, and high diffusion resistance for unexpected species and macromolecules.

U.S. Pat. No. 7,799,191B2 describes the preparation of an enzyme layer formed on the surface of an electrode that is covered by an epoxy polymer layer. The epoxy polymer layer covers the immobilized enzyme layer in order to add durability to the underlying enzyme layer, while also, in certain configurations, serves as a diffusion barrier to the internal enzyme layer.

Although these different approaches are geared towards stabilizing the enzymatic-based glucose sensor, there is still an unmet need of a stable membrane system that does not change over time. Conventional methods of enzyme immobilization within multilayered membranes still lack long term stability and operability. The current invention addresses this current problem by providing for a chemical fusing of multilayered membranes such that the chemically fused membranes impart a greater stability to the sensor signal and accuracy.

The forgoing examples of related art and limitations related therewith are intended to be illustrative and not exclusive, and they do not imply any limitations on the invention described and claimed herein. Various limitations of the related art will become apparent to those skilled in the art upon a reading and understanding of the specification below and the accompanying drawings.

### SUMMARY OF THE INVENTION

The device herein disclosed and described provides an analyte sensor, having a working electrode and a multilayered membrane disposed over the electrode. The membrane is formed by covalently attaching an outer layer comprised of an ethylenically unsaturated prepolymer to an inner layer comprised of an ethylenically unsaturated polyelectrolyte and an enzyme. The final fused membrane composition acts a sensor membrane that provides a more stable and robust system. More specifically, the multilayered membrane formed comprises a restrictive domain that controls the flux of oxygen and glucose through the membrane to the working electrode without significant drift in sensor signal.

Another aspect of the present invention is a method of making an analyte sensor, comprising the steps of disposing a sensing layer on a surface, treating the sensing layer with a coupling agent and attaching ethylenically unsaturated functional groups, and applying another layer over the sensing layer and curing the coated solution at a temperature range of between 4° C.-80° C. The membrane being prepared from a composition reaction mixture of a polyelectrolyte mixed with an enzyme and a crosslinker as a first layer that is functionalized with ethylenically unsaturated groups and chemically reacted with an outer layer comprised of an ethylenically unsaturated prepolymer.

In one embodiment of either aspect of the invention, the polyelectrolyte comprises about 1 to about 10 percent of the membrane. More specifically, the polyelectrolyte comprises about 1, about 2, about 3, about 4, about 5, about 6, about 7, about 8, about 9, about 10 percent of the membrane formed from the composition reaction mixture.

In one embodiment of either aspect of the invention, the enzyme comprises about 1 to about 10 percent of the membrane. More specifically, the enzyme comprises about 0.5, about 1, about 2, about 3, about 4, about 5, about 6, about 7, about 8, about 9, about 10 percent of the membrane formed from the composition reaction mixture.

In another embodiment of either aspect of the invention, the crosslinker comprises about 0.1 to about 5 percent of the membrane. More specifically, the crosslinker comprises about 0.1, about 0.2, about 0.3, about 0.4, about 0.5, about 0.6, about 0.7, about 0.8, about 0.9, about 1, about 1.2, about 1.5, about 2 about 2.5, about 3, about 3.5, about 4, about 4.5, about 5 percent of the membrane formed from the crosslinker composition reaction mixture.

In another embodiment of either aspect of the invention, the polyelectrolyte is comprised of carboxylic acid, hydroxy, and amino functional groups.

In another embodiment of either aspect of the invention, the ethylenically unsaturated monomer that is attached to the sensing layer is comprised of hydroxy, methacrylate, acrylate, vinyl, and ester end groups; and alkyl and ether main chain groups. More specifically, the monomer is allyl alcohol, 2-allyloxyethanol, 2-hydroxyethyl methacrylate, 3-hydroxypropyl methacrylate, glycidyl methacrylate, diethyleneglycol monomethacrylate, diethylene glycol methyl ether methacrylate, polyethylene glycol monomethacrylate, polyethylene glycol monoacrylate, allyl methacrylate, methacrylic acid, acrylic acid.

In one embodiment of either aspect of the invention, the coupling agent comprises a carbodiimide functionality.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention.

The objects, features, and advantages of the invention will be brought out in the following part of the specification, wherein detailed description is for the purpose of fully disclosing the invention without placing limitations thereon.

### BRIEF DESCRIPTION OF THE DRAWINGS

FIG. 1 shows the enzymatic oxidation of glucose with glucose oxidase.

FIG. 2 shows the reaction of enzyme with crosslinker aziridine and polyacrylic acid.

FIG. 3 shows the EDC coupling reaction of 2-hydroxyethylmethacrylate to polyacrylic acid-enzyme polymer to create an ethylenically unsaturated enzyme prepolymer composition.

FIG. 4 shows the concept of covalently attaching separate membrane layers via polymerization of their ethylenically unsaturated monomers.

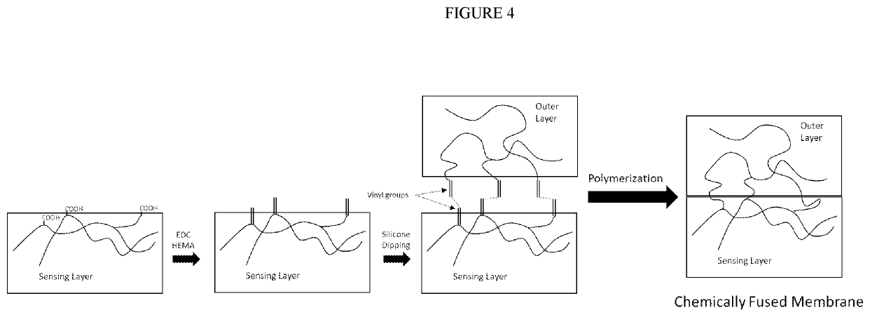

FIG. 5 shows the percentage change in sensor sensitivity of a series of sensor wires treated with EDC/HEMA in comparison to no EDC/HEMA treatment.

FIG. 6 shows a comparison of glucose response curves for sensor wires treated with EDC/HEMA and not treated with EDC/HEMA.

### DETAILED DESCRIPTION OF THE INVENTION

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail.

As used herein, the term “alkyl” refers to a single bond chain of hydrocarbons ranging, in some embodiments, from 1-20 carbon atoms, and ranging in some embodiments, from 1-8 carbon atoms; examples include methyl, ethyl, propyl, isopropyl, n-butyl, sec-butyl, isobutyl, tert-butyl, pentyl, isoamyl, hexyl, octyl, dodecanyl, and the like.

The term “analyte” as used herein, refers to a substance or chemical constituent in a biological fluid (for example, blood, interstitial fluid, cerebral spinal fluid, lymph fluid, or urine) that can be analyzed. Analytes include naturally occurring substances, artificial substances, metabolites, and/or reaction products. In some embodiments, the analyte for measurement by the sensor is glucose.

The terms “sensor” or “sensing” as used herein is a description of the component or region of a device by which an analyte can be quantified.

The term “domain” as used herein, describes regions of the membrane that may be layers, uniform or non-uniform gradients (for example, anisotropic), functional aspects of a material, or provided as portions of the membrane.

The term “silicone” as used herein, describes a composition of matter that comprises polymers having alternating silicon and oxygen atoms in the backbone. Examples include, but are not limited to, vinyl terminated polydimethylsiloxane and vinylmethylsiloxane copolymer.

The phrase “ethylenically unsaturated” as used herein, describes a composition of matter that comprises a carbon-carbon double bond that can be further reacted. Examples include but are not limited to 2-hydroxyethyl methacrylate and polyethyleneglycol dimethacrylate.

The term “HEMA” as used herein, refers to 2-hydroxyethyl methacrylate.

The term “azirdine” as used herein, refers to compounds containing one or more of the aziridine functional group, a three-membered heterocycle with one amine (—NR—) and two methylene bridges (—CR₂—). Examples include but are not limited to N,N′-(methylenedi-p-phenylene)bis(aziridine-1-carboxamide) and Trimethylolpropane tris(2-methyl-1-aziridine propionate).

The term “epoxide” as used herein, refers to compounds containing one or more of the epoxide functional group, a three-membered heterocycle with one oxygen (—O—) and two methylene bridges (—CR₂—). Examples include but are not limited to 1,4-butanediol diglycidyl ether and 4,4′-methylenebis(N,N-diglycidylaniline).

The term “prepolymer” as used herein, describes a composition of matter that comprises a monomer or system of monomers that have been reacted to an intermediate molecular mass state. This material is capable of further polymerization by reactive groups to a fully cured high molecular weight state. Examples include but are not limited to polyacrylic acid, vinylsiloxane, and polyethyleneglycol dimethacrylate.

The term “crosslinker” as used herein, refers to compounds used to connect two or more polymer chains. Examples included but are not limited to aziridines, epoxides, aldehydes, and carbodiimides.

The invention disclosed herein provides a glucose sensor membrane that solves the problems of the previous membranes both in terms of potential in vivo problems and in terms of membrane preparation in that it restricts glucose diffusion, is highly oxygen permeable, is mechanically strong, forms a crosslinked polymer network, is highly biocompatible, is stable over time, and may be prepared as a dip-coating.

The device herein disclosed and described provides an analyte sensor, having a working electrode and a multilayered membrane disposed over the electrode. The membrane is formed by covalently attaching an outer layer comprised of an ethylenically unsaturated prepolymer to an inner layer comprised of an ethylenically unsaturated polyelectrolyte and an enzyme. The final fused membrane composition acts a sensor membrane that provides a more stable and robust system. More specifically, the multilayered membrane formed comprises a restrictive domain that controls the flux of oxygen and glucose through the membrane to the working electrode without significant drift in sensor signal

Another aspect of the present invention is a method of making an analyte sensor, comprising the steps of disposing a sensing layer on a surface, treating the sensing layer with a coupling agent and attaching ethylenically unsaturated functional groups, and applying another layer over the sensing layer and curing the coated solution at a temperature range of between 4° C.-80° C. The membrane being prepared from a composition reaction mixture of a polyelectrolyte mixed with an enzyme and a crosslinker as a first layer that is functionalized with ethylenically unsaturated groups and chemically reacted with an outer layer comprised of an ethylenically unsaturated prepolymer.

Embodiments of the invention include a sensor having a plurality of layered elements including an analyte limiting membrane comprising a transition metal cured crosslinked silicone. Such polymeric membranes are particularly useful in the construction of electrochemical sensors for in vivo use, and embodiments of the invention include specific biosensor configurations that incorporate these polymeric membranes. The membrane embodiments of the invention allow for a combination of desirable properties including: permeability to molecules such as glucose over a range of temperatures, good mechanical properties of use as an outer polymeric membrane, and good processing properties for in situ preparation on a substrate. Consequently, glucose sensors that incorporate such polymeric membranes show an enhanced in vivo performance profile.

The ethylenically unsaturated silicone prepolymer may comprise about 40 to about 90 percent of the membrane. More specifically, the ethylenically unsaturated silicone prepolymer may comprise about 40, about 45, about 50, about 55, about 60, about 65, about 70, about 75, about 80, about 85 or about 90 percent of the membrane formed from the silicone composition reaction mixture.

The ethylenically unsaturated hydrophilic monomer may be comprised of hydroxy, alkoxy, epoxy, vinyl, and carboxylic acid end groups; and alkyl and ether main chain groups. More specifically, the monomer may be allyl alcohol, 2-allyloxyethanol, 2-hydroxyethyl methacrylate, 3-hydroxypropyl methacrylate, glycidyl methacrylate, diethyleneglycol dimethacrylate, diethylene glycol methyl ether methacrylate, polyethylene glycol monomethacrylate, polyethylene glycol dimethacrylate, allyl methacrylate, methacrylic acid, acrylic acid. Further, the ethylenically unsaturated monomer may comprise about 2 to about 30 percent of the membrane. More specifically, the ethylenically unsaturated monomer may comprise about 2, about 4, about 6, about 8, about 10, about 12, about 15, about 20, about 24, about 28 or about 30 percent of the membrane formed from the composition reaction mixture.

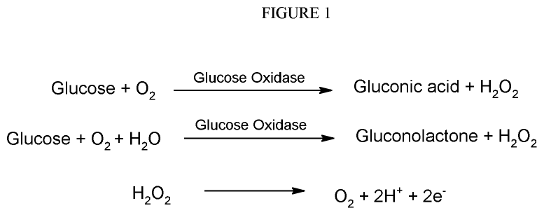

The continuous glucose monitoring system described herein is inserted underneath the skin with a small needle. The needle is removed and the sensor resides in the interstitial fluid and comes in direct contact with fluid containing glucose. The glucose permeates through the sensor membrane and reacts with glucose oxidase generating hydrogen peroxide that is then detected amperometrically (FIG. 1). Similar systems are described in In Vivo Glucose Sensing, Cunningham, D. D., Stenken, J. A., Eds; John Wiley & Sons, Hoboken, N.J., 2010.

The unexpected result is that when a hydrophilic enzyme polymer layer is formed with a methacrylate functional group creating a prepolymer, a second hydrophobic polymeric layer can be covalently attached to the enzyme layer through a polymerization reaction to provide a more stable and robust sensing system that has less drift than a standard multilayered membrane system that is not covalently bound to the other. More specifically, the ability to connect two different polymer layer phases (i.e., hydrophilic and hydrophobic) via a polymerization reaction was unexpected and had not previously been done.

### EXAMPLES

### Example 1

### Preparation of a Chemically Fused Membrane Glucose Sensor

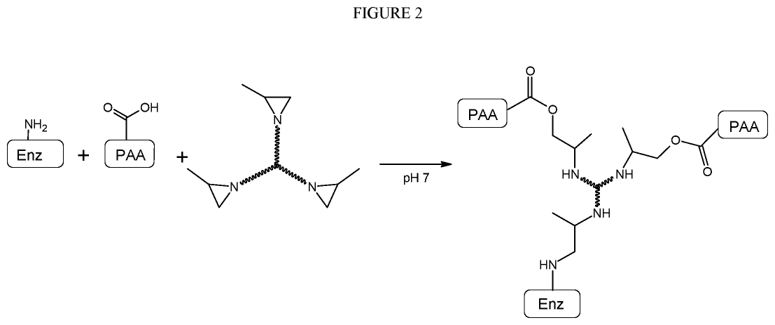

Preparation of an enzyme membrane dipping solution (FIG. 2). Polyacrylic acid (PAA, MW 400,000, 10 g) was added to phosphate buffered saline (pH 7.0, 50 mM, 90 mL) and stirred for 16 h at room temperature. In a separate container, 0.50 g glucose oxidase (GOX) was added to 5.00 g of pH 7.0 PBS. The solution was mixed with a speed mixer at 1400 rpm for 20 sec. Polyacrylic acid solution (5.00 g) was added to the GOX solution and mixed using a speed mixer set at 1400 rpm for 20 s. Trimethylolpropane tris(2-methyl-1-aziridine propionate) (0.1 g) was added into the GOX/PAA solution and mixed with a speed mixer set at 1400 rpm for 20 s.

Dipping of enzyme solution on wire. Three 60 mm platinum wires were attached to a glass microscope slide such that 10 mm was exposed at the distal end of the wires. Using a dip coater the wires were dipped and dried until the wire OD+coating=85 μm thick (wire OD−Coating=2.5 μm). The slide with wires was placed in oven at 60° C. to cure for 2 hours.

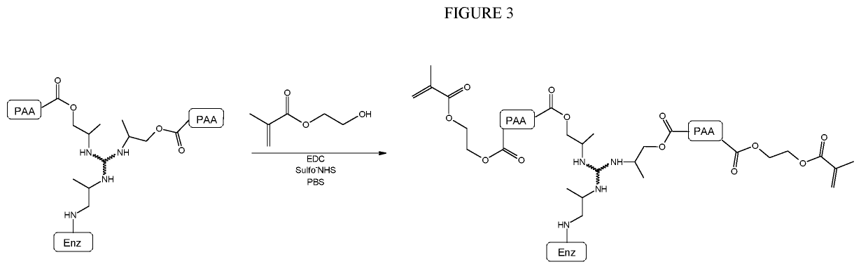

Preparation of 1-Ethyl-3-(3-dimethylaminopropyl) carbodiimide (EDC) coupling solution (FIG. 3). 1-Ethyl-3-(3-dimethylaminopropyl) carbodiimide (156 mg) and phosphate buffered saline (pH 7.0, 50 mM, 10 mL) were added to a container and the mixture was vortexed. Sulfo-N-hydroxy succinimide (434 mg) was added along with 2-hydroxyethylmethacrylate (124 μL) and the mixture was stirred for 5 s with a vortex mixture.

Dipping of Enzyme coated wire into EDC solution. A microscope slide with 3 enzyme coated wires with 4 mm of the distal end of the wires exposed were dipped into the EDC solution for 1.5 hours and then transferred to a phosphate buffered saline (PBS) solution (pH 7.4, 50 mM, 10 mL). The wires were soaked in the PBS solution for 5 min and then transferred to a 60° C. oven and dried for 20 minutes.

Preparation of Silicone Dipping Solution

Using two part oleophilic reprographic silicone from Gelest, Inc. (Morrisville, Pa.), 7.03 g of part A (vinyl terminated polydimethylsiloxane) was mixed with 2.97 g of 2-hydroxyethyl methacrylate containing 2% diethyleneglycol dimethacrylate and 1.00 g of part B (methylhydrosiloxane-dimethylsiloxane copolymer, trimethylsiloxane terminated with vinyl, methyl modified silica). The mixture was speed mixed for 40 seconds.

Dipping of EDC-treated wires into silicone solution. The silicone dipping solution was transferred to a 40 mL plastic cup and placed under a dipping arm. The EDC-treated wires were dip-coated with the silicone solution until a thickness of approximately 15μ was achieved. The coated wire was heated in an oven at 60° C. for 16 hours.

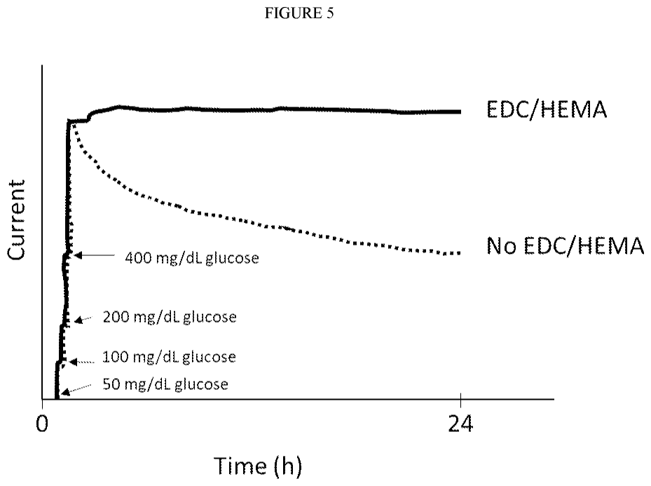

Testing of an EDC treated wire (FIG. 5). The EDC treated sensor wire that was coated with a silicone outer membrane was evaluated as part of a two electrode electrochemical system. The counter and reference electrode was an iridium oxide coated wire. For comparison, two sets of wire types were prepared: one that was not EDC/HEMA-treated; and one that was EDC/HEMA-treated. For each wire, the current was measured amperometrically and the electrochemical response was measured as a function of glucose concentration. The concentration range of 0-400 mg/dL glucose was evaluated.

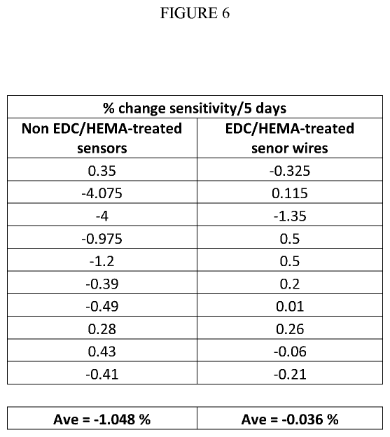

The glucose response in vitro demonstrates the signal stability ability of the EDC/HEMA treated membrane: without the membrane the average sensor sensitivity decreases by 1.3% over 5 days, whereas with EDC/HEMA treatment the average sensor sensitivity decreases by 0.036% (FIG. 6).

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

We claim:

1. An analyte sensor, comprising:
a working electrode; and
a multilayered membrane disposed over said electrode, said membrane formed from a reaction mixture comprising:
a first sensing layer of an ethylenically unsaturated polyelectrolyte prepolymer and
a subsequent flux limiting layer of an ethylenically unsaturated prepolymer, wherein said layers formed from said composition reaction mixture are covalently attached to each other.

2. The sensor of claim 1, wherein the sensing layer comprises an enzyme.

3. The sensor of claim 2, wherein the enzyme is glucose oxidase, glucose dehydrogenase, catalase or 3-hydroxybutyrate dehydrogenase.

4. The sensor of claim 1, wherein the polyelectrolyte is a carboxylic acid.

5. The sensor of claim 4, wherein the carboxylic acid is polyacrylic acid.

6. The sensor of claim 1, wherein the sensing layer is formed through a crosslinking reaction. The sensor of claim 6, wherein the crosslinker is an aziridine or epoxide.

8. The sensor of claim 1, wherein the membrane is configured and arranged to reduce flux of an analyte to the sensing layer.

9. The sensor of claim 1, wherein the flux limiting layer comprises an ethylenically unsaturated silicone prepolymer.

10. The sensor of claim 1, further comprising a biocompatible layer disposed over the multilayer membrane.

11. The sensor of claim 1, wherein the membrane is configured and arranged to reduce flux of at least one interferent to the sensing layer.

12. The sensor of claim 1, wherein the sensor is adapted for implantation of at least a portion of the sensor in an animal.

13. The sensor of claim 1, wherein the sensor is adapted for subcutaneous implantation of at least a portion of the sensor in an animal.

14. The analyte sensor according to claim 1, wherein said ethylenically unsaturated monomer is comprised of functional groups consisting of hydroxy, ethoxy, methoxy, ethylene oxide, propylene oxide, methacrylate, acrylate, and carboxylic acids.

15. The analyte sensor according to claim 1, wherein said ethylenically unsaturated monomer is 2-hydroxyethyl methacrylate, 3-hydroxypropyl methacrylate, glycidyl methacrylate, diethyleneglycol dimethacrylate, diethylene glycol methyl ether methacrylate, polyethylene glycol monomethacrylate, polyethylene glycol dimethacrylate, allyl methacrylate, methacrylic acid, acrylic acid, allyl alcohol, 2-allyloxyethanol.

16. A method of making a sensor, comprising the steps of:
disposing a first layer on a substrate; wherein said first layer is formed in a crosslinking reaction;
chemically modifying said first layer with ethylenically unsaturated groups; and
disposing a subsequent layer comprising an ethylenically unsaturated prepolymer;
wherein said subsequent layer is formed in a chain-growth polymerization reaction.

17. The method of claim 16, wherein the crosslinking reaction is between a carboxylic acid and an aziridine.

18. The method of claim 16, wherein the crosslinking reaction is between a carboxylic acid and an epoxide.

19. The method of claim 16, wherein chain-growth polymerization reaction is a platinum cured hydrosilyation reaction or a free radical reaction.

20. The method of claim 19, wherein the free radical reaction is initiated by a photoinitiator or a thermal initiator.
